(19) United States
(12) Patent Application Publication
(10) Pub. No.: US 2025/0214758 A1
(43) Pub. Date: Jul. 3, 2025

(54) PACKAGING AND/OR LABELING FOR A MEDICAL CONTAINER

(71) Applicant: Fresenius Kabi USA, LLC, Lake Zurich, IL (US)

(72) Inventors: Tyler O'NEILL, Wilson, NC (US); James PROCTOR, Wilson, NC (US)

(21) Appl. No.: 18/396,756

(22) Filed: Dec. 27, 2023

(51) Int. Cl. B65D 77/02; A61M 5/00; B65B 3/00; B65B 5/04; B65B 53/02; B65B 61/02; B65D 75/00

(52) U.S. Cl. CPC B65D 77/02; A61M 5/002; B65B 3/003; B65B 5/04; B65B 53/02; B65B 61/025; B65D 75/004; A61M 2207/00; B65B 2220/16; B65D 2203/02

(57) ABSTRACT

An injection device may include a container body configured to contain a pharmaceutical material and a film disposed over the container body. The film may have a beveled or rounded corner. The beveled or rounded corner may have a different shape than two other corners of the film. The film may be a label having indicia pertaining to the pharmaceutical material. The container may include a plunger rod extending through the container body and being configured to push the pharmaceutical material out of the container body. The injection device may be received in a shell for packaging. The beveled or rounded corner of the film may limit or prevent the film from peeling and/or curling up from the container body to ensure that the injection device properly fits into the shell.

### TECHNICAL FIELD

The disclosure relates generally to devices, systems, and methods of manufacturing medical containers, and more particularly relates to improved labeling and/or packaging.

### BACKGROUND

Medical containers, such as syringes and/or cartridges, are adapted for storing and/or transporting a pharmaceutical material. Various films may be applied to the medical container, for example, to secure an adapter to the medical container and/or identify the pharmaceutical material.

### SUMMARY

The inventors recognize a problem that happens when the film on the medical container peels and/or curls up at least partially separating from the medical container. More specifically, the inventors recognize that the curl up of the film may prevent the syringe from fitting into a secondary container and/or cause damage to the syringe to when being inserted in the secondary container when inserted by a robot. The curling up of the film may further reduce the aesthetic of the product, possibly causing the medical container to be rejected and/or discarded creating waste.

One or more of the foregoing needs are met by the various embodiments as disclosed herein.

A first aspect of the present disclosure is directed to an injection device comprising: a container body configured to contain a pharmaceutical material; and a film disposed over the container body, wherein the film has a beveled corner.

In some embodiments, the injection device may have one or more of the following features. The film is a label having indicia pertaining to the pharmaceutical material. The film is heat shrunk over the container body. The container includes a plunger rod extending through the container body and being configured to push the pharmaceutical material out of the container body. The injection device includes an adapter configured to connect to an external device, wherein the film extends over the adapter to fix the adapter to the container body. The container body includes a tip that extends through the adapter, and the adapter has a threaded inner surface. The container body is made of a glass and the adapter is made of a plastic. The injection device further includes a plug attached to the adapter and configured to seal a tip of the container body. The film extends over the plug. The film is asymmetric relative to at least one of a vertical axis and/or a lateral axis. The beveled corner extends at an angle of about 30 degrees to about 45 degrees relative to an axis of the film. The beveled corner has a length of about 6 mm to about 10 mm. The beveled corner extends at an angle of about 45 degrees relative to a vertical axis of the film and has a length of about 8 mm. The beveled corner may limit or prevent peeling and/or curling of the film.

A second aspect of the present disclosure is directed to a system comprising: the injection device as discussed above; and a shell receiving the container.

In some embodiments, the injection device may have one or more of the following features. The shell is tubular. The shell includes a first body, a second body, and a frangible portion attaching the first body and the second body. The frangible portion is configured to be broken by rotating the first body relative to the second body. The container body is inserted into the shell with the beveled corner being a leading corner.

A third aspect of the present disclosure is directed to a system comprising: a container and a tubular shall receiving the container. The container including: a container body containing a pharmaceutical material, and a film disposed over the container body, wherein the film is asymmetric relative to at least one of a vertical and/or a lateral axis.

In some embodiments, the system may include one or more of the following features. The film has a first corner is rounded with a first radius of curvature and at least two second corners are rounded with a second radius of curvature, wherein the first radius of curvature is different than the second radius of curvature. The first radius of curvature is about 6 mm, and the second radius of curvature is about 3 mm.

A fourth aspect is directed to a system comprising: a container including: a container body containing a pharmaceutical material and having a tip, a heat shrink film disposed over at least a portion of the container body, and a plunger rod extending through the container body and being configured to push the pharmaceutical material out of the container body; an adapter configured to connect to an external device, wherein the heat shrink film extends over the adapter to fix the adapter to the container body; a plug received in the adapter and sealing the tip of the container body; and a tubular shell receiving the adapter and the container, wherein the heat shrink film has a beveled corner configured to limit or prevent and/or curling, and the beveled corner extends at an angle of about 45 degrees relative to an axis of the heat shrink film and has a length of about 8 mm.

In some embodiments, the system may include one or more of the following features. The heat shrink film is asymmetric relative to at least one of a vertical and/or a lateral axis. The heat shrink film extends over the plug to fix the plug to the adapter.

A fifth aspect of the present disclosure is directed to a method of assembly comprising: filling a container body with a pharmaceutical material; inserting a plunger rod into the container body; attaching a film to the container body, wherein the film has a beveled corner; and inserting the container body into a shell with the beveled corner being a leading corner.

In some embodiments, the method may include one or more of the following features. Attaching the film to the container body includes heat shrinking the film. Attaching the film to the container body includes securing an adhesive to the container body. The method further includes fixing an adapter around a tip of the container body by attaching the film to the adapter.

### BRIEF DESCRIPTION OF THE DRAWINGS

Specific embodiments of the present disclosure are described below in the detailed description by way of example only and with reference to the accompanying drawings, in which:

FIG. 1 illustrates a side view of a packaging system including a medical container.

FIG. 2 illustrates a side view of the medical container of FIG. 1.

FIG. 3 illustrates an exploded view of components of the medical container of FIGS. 1-2.

FIG. 4 illustrates an assembled view of components of the medical container of FIGS. 1-3.

FIG. 5 illustrates a cross-sectional view of the medical container of FIGS. 1-4.

FIG. 6 illustrates a side view of an alternative embodiment of the medical container of FIGS. 1-5.

FIG. 7 illustrates a first embodiment of a film of the medical container of FIGS. 1-6.

FIG. 8 illustrates a second embodiment of the film of the medical container of FIGS. 1-6.

The same reference numbers are used in the drawings and the following detailed description to refer to the same or similar parts.

### DETAILED DESCRIPTION

The present disclosure relates to an improved film for medical containers that improves consistency in manufacturing medical containers. The film may be a label wrapped around the container to provide information of a pharmaceutical material disposed in the container. The film may include one or more corners or edges that may limit or prevent the film from peeling and/or curling up from the container. The one or more corners of the film may be a beveled and/or rounded to limit or prevent the edge from being lifted from the container by and/or abutting an edge of an object, for example when the container is being inserted into a secondary packaging with a tight clearance. In some embodiments, a corner of the film may be beveled at an angle of about 40 degrees to about 65 degrees relative to an upper surface. In some embodiments, a corner of the film may have a larger radius of curvature of about 6 mm. The improved adherence of the film may further ensure consistent aesthetics of the medical container, by enhancing the user experience and/or reducing waste of rejection. The film may further be applied to the container to fix a Luer adaptor and/or plug to the container preventing relative rotation and longitudinal movement during storage and/or handling.

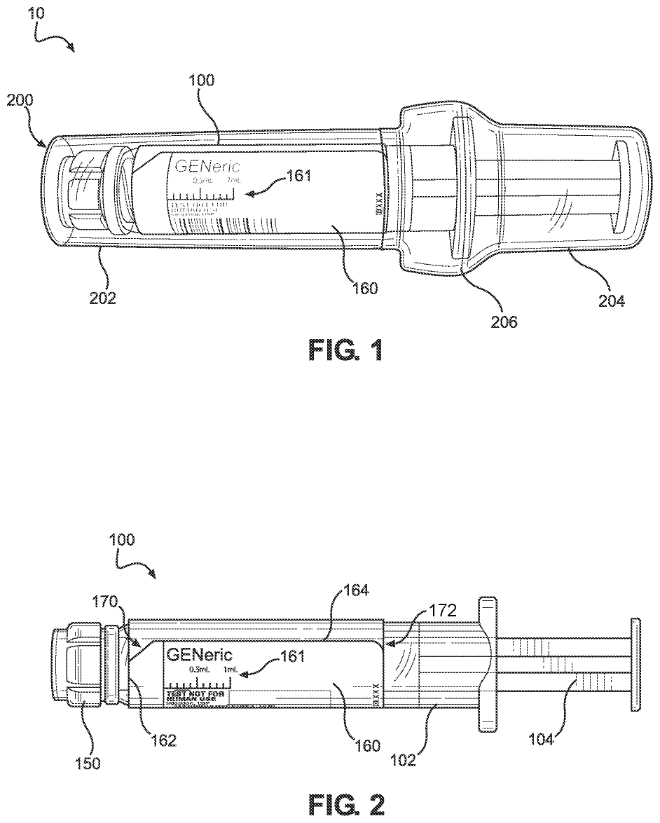

FIGS. 1-5 illustrate an embodiment of a system 10 including a packaged container. The system 10 may include a container 100 containing a pharmaceutical material, a film 160 on the container 100, and a shell 200 receiving the container 100.

The container 100 may include a container body 102 and a plunger rod 104. The container body 102 may have a barrel 106 extending from a proximal portion to a distal portion along a longitudinal direction. The container body 102 may further have a tip 108 at the distal portion of the barrel 106 and a flange 110 at the proximal portion of the barrel 106. The flange 110 may extend radially outwards from the proximal end of the container body 102 to enable stability and handling of the container body 102. The flange 110 may have a substantially oval shape with two flats, oppositely positioned sides and two arcuate, oppositely positioned sides. Alternatively, the flange 110 may have a different shape, such as a substantially circular shape. The flange 110 may define a proximal opening sized to receive the plunger rod 104. The barrel 106 may be tubular having an inner surface extending along the longitudinal direction to define a chamber 112. The chamber 112 may be configured to receive, store, and/or mix the pharmaceutical material for dispensing through a distal opening of the tip 108. The plunger rod 104 may have a plunger stopper (not shown) at a distal portion to define the proximal-most extent to which the material can fill the chamber 112. The plunger stopper may have an enlarged, substantially cylindrical body. The plunger stopper may be made of a flexible elastomeric material (e.g., rubber) and generally conform to the shape of the chamber 112 to prevent leakage of the material proximally out of the chamber 112. As the plunger stopper moves distally through the chamber 112, the plunger stopper may push the material out of the chamber 112 through the tip 108. A proximal portion of the plunger rod may extend proximally from the container body 102 to be actuated by a user.

The container body 102 and/or plunger rod 104 may be made of glass or a plastic material, such as polyethylene terephthalate (PET), polypropylene (PP), polycarbonate (PC), cyclic olefin polymer (COP), cyclic olefin copolymer (COC), or other material. However, the invention may be particularly suitable for embodiments where the container body 102 is made of glass, which provides an inert container that minimizes chemical or biological degradation of the material and may extend the expiration. The container body 102 may be translucent and/or transparent to enable visual inspection of the pharmaceutical material. The container 100 may be a syringe and/or a cartridge. In some embodiments, the container may be a pre-filled syringe packaged containing the pharmaceutical material and removable from the shell 200 ready-to-use.

The pharmaceutical material may include an active ingredient of a pharmaceutical formulation of a therapeutic agent, a diagnostic agent, a nutrient, or combinations thereof. Examples of therapeutic agents include, but are not limited to, anti-infectives, anesthetics, analgesics, anticoagulants, chemotherapeutics, hormones, antihypertensives, anti-inflammatories, antiemetics, bronchodilators, adrenergics, immunoglobulins, antipsychotics, antidepressants, and combinations thereof. Examples of diagnostic agents include, but are not limited to, X-ray, magnetic resonance imaging (MRI), and ultrasound contrast agents, cholecystokinetics, vasodilators, and combinations thereof. Examples of nutrients include, but are not limited to, salts, carbohydrates, minerals, vitamins, lipids, and combinations thereof.

In some embodiments, the active ingredient is a compound useful for pain management, muscle relaxation, sedation, and/or anesthesia. In certain embodiments, the active ingredient is an opioid, a benzodiazepine, beta blocker, or an α-2-adrenergic receptor agonist. In particular embodiments, the active ingredient is morphine, hydromorphone, hydrocodone, oxycodone, oxymorphone, codeine, buprenorphine, naloxone, naltrexone, fentanyl, remifentanil, sufentanil, alfentanil, meperidine, rocuronium, vecuronium, midazolam, lorazepam, diazepam, neostigmine, atropine, glycopyrrolate, dexmedetomidine, cisastracurium, ropivacaine, lidocaine, propofol, ketamine, succinylcholine, or a combination of the foregoing.

In other embodiments, the active ingredient is moxifloxacin, linezolid, levofloxacin, levetiracetam, vancomycin, cefepime, aztreonam, cefoxitin, ceftriaxone, cefazolin, cefotaxime, ceftazidime, gentamicin, oxacillin, nafcillin, penicillin, cefuroxime, ticarcillin, clavulanic acid, piperacillin, tazobactam, azithromycin, meropenem, ertapenem, tigecycline, micafungin, metronidazole, fluconazole, itraconazole, posaconazole, heparin, enoxaparin, dalteparin, theophylline, acetaminophen (paracetamol), ibuprofen, acetylcysteine, decitabine, azacitidine, docetaxel, pemetrexed, palonosetron, aprepitant, fosaprepitant, famotidine, amiodarone, nitroglycerin, nicardipine, clevidipine, dobutamine, esmolol, labetalol, metroprolol, somatropin, liraglutide, abaloparatide, semaglutide, teriparatide, degarelix, sumatriptan, epinephrine, ephedrine, vasopressin, methotrexate, testosterone, hydroxyprogesterone, or a combination of the foregoing.

The shell 200 may be a tubular packaging composed of a first body 202 and a second body 204 configured to be separated to access the container 100. One or both of the first body 202 and the second body 204 may be a tubular body. The first body 202 may contain at least a portion of the container body 102 including the barrel 106 and/or the flange 110. The second body 204 may contain the proximal portion of the plunger rod 104. The first body 202 and the second body 204 may be attached at a frangible portion 206 allowing the second body 204 to be separated from the first body 202. The frangible portion 206 may include bridges integrally attaching the first body 202 and the second body 204. The frangible portion 206 may, additionally or alternatively, be another component such as a ring and/or a film surrounding the first body 202 and the second body 204. In some embodiments, the frangible portion 206 may be broken by rotating or twisting the second body 204 relative to the first body 202. The container 100 may be then removed through an opening of the first body 202 by the user grasping the proximal portion of the plunger rod 104 and/or the flange 110. The shell 200 may be made of a hard plastic for diversion deterrent. The shell 200 may also be clear or translucent to allow for visibility of the pharmaceutical material. The shell 200 may additionally or alternatively allow for visibility and/or scanning of one or more indicia 161 on the film 160 when the container 100 is contained in the shell 200. For example, the shell 200 may be made of polypropylene. The shell 200 may, additionally or alternatively, including a second film (not shown). The second film may include one or more layers of a paper and/or plastic secured to the shell with an adhesive and/or heat shrink. The second film may include indicia and/or be a label, as similarly disclosed for the film 160 (the disclosure of which is incorporated herein for sake of brevity). The second film may be positioned on the second body 204, such that the label does not block the indicia 161 on the film 160 over the container body 102. The shell 200 may take up less room than a blister pack in the locked compartments of automated dispensing cabinets (ADCs).

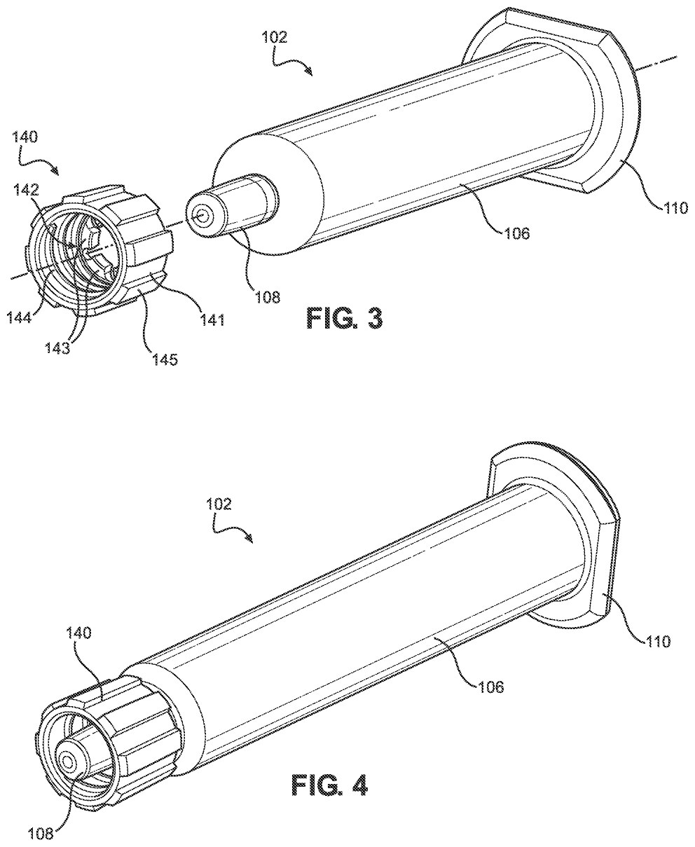

As illustrated in FIGS. 3-5, an adapter 140 may be disposed at an end of the container 100 for engagement with an external device, such as a syringe needle, a medical transport line, or another container (not shown). The adapter 140 may be configured to seal assemblies of medical devices and provide protection against the contamination of the medical liquid product and enable handling of liquid products, in particular for a parenteral administration to a patient which is carried out via a perfusion device, as often in hospitals or in emergency situations.

The adapter 140 may have a longitudinal wall 141 and a ring 142. The ring 142 may be on a proximal end of the adapter 140 and be configured to be mounted onto the tip 108 of the container body 102 by friction. As illustrated, the ring 142 may be formed by one or more projections 143 extending inwardly, for example, in the form of a discontinuous annular bulge extending radially inwardly of a proximal region of the ring 142. Alternatively, the one or more projections 143 may be a continuous annular bulge configured to expand radially outwardly. The one or more projections 143 may be made of a resiliently flexible material (e.g., a plastic) configured to expand slightly radially in the outward direction with longitudinal pressure applied to the ring 142 against the tip 108. The ring 142 may have a tapered shape configured to wedge the one or more projections 143 in order to expand and frictionally engage the one or more projections 143. The tip 108 may extend distally past a distal end of the adapter 140.

A plug 150 may be secured to the adapter to seal access to the tip 108 of the container body 102. The plug 150 may be removably mounted on the longitudinal wall 141 by friction-fit or by screwing. The plug 150 may have a distal portion 151 having a threaded outer surface 152. The distal portion 151 of the plug 150 may be received in the longitudinal wall 141, such that the thread outer surface 152 may engage a threaded inner surface 144 of the longitudinal wall 141 distal of the ring 142. The plug 150 may further have a proximal portion 153 configured to be handled by the user. To facilitate handling and relative rotation (twisting), the outer surface of the proximal portion 153 of the plug 150 may have a plurality of ribs 154 extending longitudinally, and the outer surface the longitudinal wall 141 of the adapter 140 may have a plurality of ribs 145 extending longitudinally. The adapter 140 and/or the plug 150 may be made of a plastic material and/or rubber.

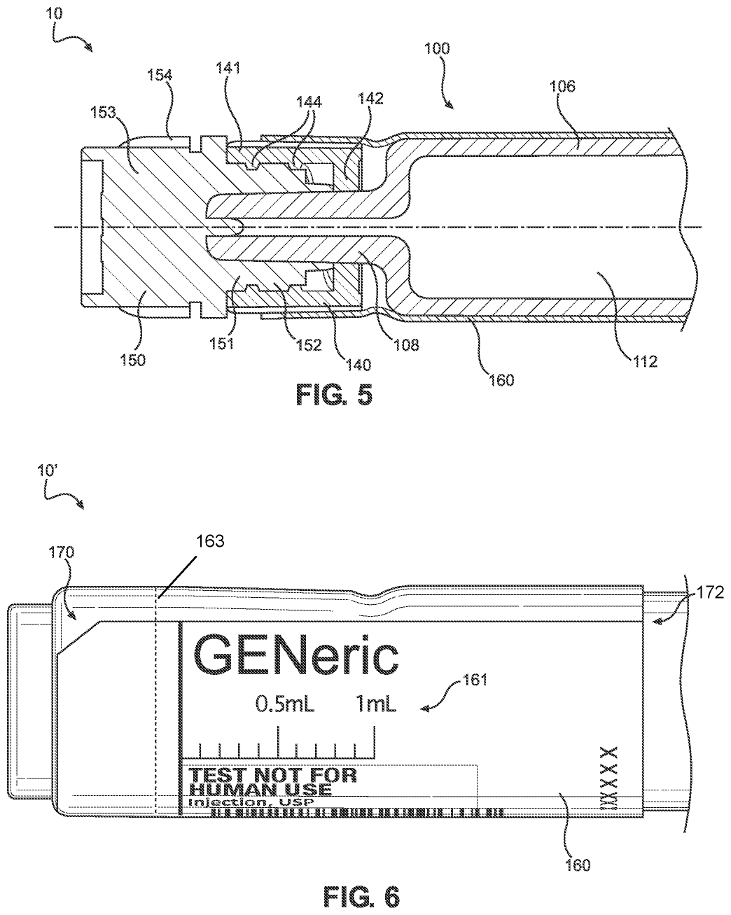

The film 160 may cover at least part of the container body 102. As further illustrated in FIG. 5, the film 160 may further cover at least a part of the adapter 140 to fix the adapter 140 to the container body 102. For example, the film 160 may at least partially overlap and/or extend continuously over the outer surface of the container body 102 and the outer surface of the adapter 140 to rotationally and translationally fix the adapter 140 to the container body 102. For example, the film 160 may be attached to the container body 102 over a length of at least 5 mm and be attached to the outer surface of the longitudinal wall 141 of the adapter 140 for a length of at least 5 mm. The fixation of the adapter 140 to the container body 102 may allow the user to grasp the outer surface of the adapter 140 to screw the plug 150 from the adapter 140 without rotation of the container body 102 relative to the adapter 140. In some embodiments of the system 10, the film 160 may not extend past the distal end of the adapter 140.

In some embodiments of the system 10′ as illustrated in FIG. 6, the film 160 may cover the adapter 140 and the plug 150 to fix the adapter 140 and the plug 150 to the container body 102 in a stored configuration. For example, the film 160 may extend continuously and/or at least partially overlap the outer surface of the container body 102, the outer surface of the longitudinal wall 141 of the adapter 140, and the outer surface of the proximal portion 153 of the plug 150. The film 160 may have a frangible portion 163 including along its length configured to be broken when the plug 150 is rotated or twisted relative to the adapter 140 in order to remove the plug 150 and administer the medication. For example, the frangible portion 163 may be a perforated circumferential line on a distal portion of the film 160 longitudinally between at least a portion of the adapter 140 and at least a portion of the plug 150. The frangible portion 163 of the film 160 may provide a tamper evident feature for opening the container 100.

In some embodiments, at least a portion of the film 160 may be made of a paper and/or a plastic. For example, an adhesive (e.g., glue) may be applied to an inner surface of the film 160 to secure the film 160 to the container body 102, the adapter 140, and/or the plug 150. In some embodiments, at least a portion of the film 160 may be made of a heat shrinkable material, and the film 160 may be heat shrunk onto the container body 102. For example, the film 160 may be at least partially made of a thermoplastic material selected from the group consisting of polyvinyl chloride (PVC), polyethylene terephthalate (PET), oriented polystyrene (OPS), oriented polypropylene (OPP), polylactic acid (PLA) and mixtures thereof. In some embodiments, the film 160 may be a single layer secured with the adhesive and/or heat shrinking. In some embodiments, the film 160 may be a composite, for example including an inner layer of a paper and/or a plastic and an outer layer of a heat shrinkable material. The heat shrinkable layer may sandwich and/or imprison the paper and/or plastic layer against an outer surface of the container body 102, the adapter 140, and/or the plug 150. The heat shrinkable material may secure the paper and/or plastic layer while being translucent and/or transparent to enable visibility of the paper and/or plastic layer that may serve as a label including the one or more indicia 161. The heat shrinkable material may additionally or alternatively serve as a label including the one or more indicia 161.

The indicia 161 may be on one or more layers of the film 160 and include text, pictures, images, markings, and/or representations. The indicia 161 may indicate one or more of a name of the pharmaceutical material, a brand name of the pharmaceutical material, a formulation of the pharmaceutical material, a batch number, a volume, a dose, a graduation, a warning, a fill date, an expiration date, directions, and/or instructions for use (IFU). The indicia 161 may, additionally or alternatively, include a marking representing data in machine-readable form, such as a linear barcode, a radio frequency identification tag, and/or a QR code. The indicia 161 may be printed on the film 160.

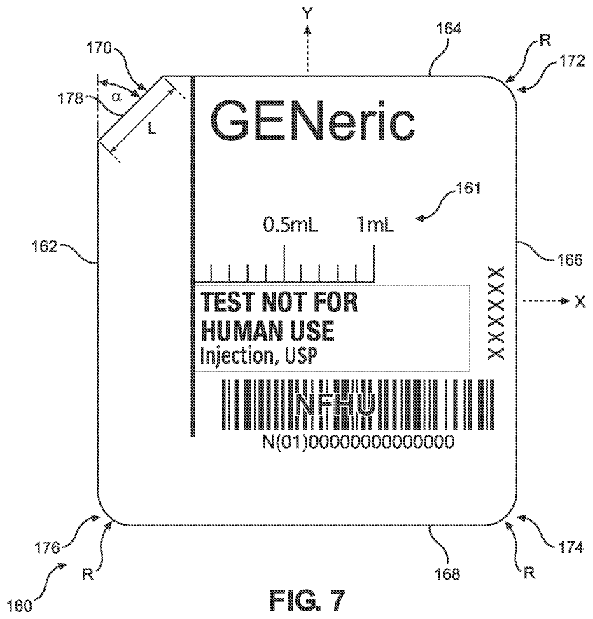

As further illustrated in FIG. 7, the film 160 may have a first side or edge 162, a second side or edge 164, a third side or edge 166, and a fourth side or edge 168. The first side 162 and the third side 166 may extend circumferentially at least partially around the container body 102 when the film 160 is attached to the container 100. The first side 162 may be proximate of the tip 108 of the container body 102 and/or the adapter 140 (e.g., closer than the third side 166). The second side 164 and the fourth sides 168 may extend along the longitudinal axis of the container body 102 when the film 160 is attached to the container 100. The film 160 may further have a first corner or edge 170 joining the first side 162 and the second side 164, a second corner or edge 172 joining the second side 164 and the third side 166, a third corner or edge 174 joining the third side 166 and the fourth side 168, and a fourth corner or edge 176 joining the fourth side 168 and the first side 162. The first side 162 and the third side 166 may extend along or parallel to a vertical axis Y, and the second side 164 and the fourth side 168 may extend along a lateral axis X, as illustrated in FIG. 7. The sides 162-168 may be flat or straight and extend at least a majority of a dimension of the film 160 along the lateral axis X or the vertical axis Y. The film 160 may substantially define a square or rectangular shape when in a planar configuration. The corners 170-176 may be sharp, rounded, and/or beveled (as discussed herein).

At least one of the corners 170-176 may have a shape that limits or prevents the film from peeling and/or curling up relative to the container body 102. The at least one of the corners 170-176 may be the first corner 170 that is proximate of the container body 102 and/or the adapter 140 relative to the second corner 172. The first corner 170 may be a leading corner of the film 160 when the container 100 is being inserted into the first body 202 of the shell 200. Thus, the shape of the first corner 170 may limit or prevent the film from peeling and/or curling up relative to the container body 102, e.g., when contacting a surface of the first body 202 of the shell 200 and/or during the application process (e.g., heat shrinking) of the film 160. The shape of the first corner 170 may ensure that the container 100 properly fits into the first body 202 of the shell 200 by ensuring that the container 100 has a width that is smaller than the opening of the first body 202 of the shell 200. The first corner 170 may also ensure consistent aesthetics of the container 100 to reduce discarding and/or waste. The shape of the first corner 170 may be different from one or more of the other corners 172-176, thus the film 160 may be asymmetric relative to at least one of the lateral axis X and/or the vertical axis Y, as illustrated in FIG. 7.

In some embodiments, as illustrated in FIGS. 1 and 2, the film 160 may be sized to have a length (along the vertical axis Y) that is longer than a circumference of the container body 102, such that the film 160 is wrapped completely around the container body 102, and the second side 164 overlaps and covers the fourth side 168. In this configuration, the fourth corner 174 may be covered by a portion of the film 160 and sandwiched against the container body 102, and thus protected. In these embodiments, the first corner 170 may be the only exposed leading corner thus be provided a different shape than the second corner 172, the third corner 174, and the fourth corner 176.

In some embodiments (not shown), the film 160 may sized to have a length (along the vertical axis Y) that is equal to or less than the circumference of the container body 102. Therefore, the fourth corner 176 may be uncovered and exposed. Therefore, the fourth corner 176 may have the same shape as the first corner 170 because it is a leading corner that is exposed to the first body 202 of the shell 200 during insertion. Thus, the first corner 170 and the fourth corner 176 may have a different shape than each of the second corner 172 and the third corner 174, which may have the same shape.

As further illustrated in FIGS. 7, the first corner 170 may be beveled to limit or prevent peeling and/or curling from the container body 102, as discussed herein. The first corner 170 may include a side or edge 178 extending between the first side 162 and the second side 164. The side 178 may be flat or straight, and the connection of the end of the side 178 to the first side 162 and the second side 164 may be at a sharp angle. The side 178 may extend a length L at an angle α relative to one of the lateral axis X or the vertical axis Y. The angle α may be about 30 to about 45 degrees relative to one of the lateral axis X or the vertical axis Y. In some embodiments, the angle α may be about 30 to about 45 degrees relative to the vertical axis Y. The length L of the side 178 may be about 3 to about 13 mm or any range therein, e.g., about 4 mm to about 12 mm, about 5 mm to about 11 mm, about 6 mm to about 10 mm, or about 7 mm to about 9 mm. The length L of the side 178 may be about 8 mm. For example, in a preferred embodiment, the angle α may be about 45 degrees relative to the vertical axis Y and the length L of the side 178 may be about 8 mm to optimize the performance of the film 160.

As further illustrated in FIG. 7, two or more of the other corners 172-176 may have the same shape as each other (e.g., rounded), but different than the shape of the first corner 170 (e.g., beveled). In some embodiments, each of the corners 172-176 may have the same shape as each other (e.g., rounded), but different than the shape of the first corner 170 (e.g., beveled). For example, two or more or each of the corners 172-176 may be defined by a rounded shape having the same radius of curvature R, as illustrated in FIG. 7. The radius of curvature R of at least two or more of the other corners 172-176 may be about 1 mm to about 5 mm, about 2 mm to about 4 mm, or about 3 mm. In other embodiments (not shown), the two or more or each of the corners 172-176 may form a sharp angle, for example 90 degree angles. The shape of the two or more of the corners 172-176 may be easier to produce and provide sufficient adhesion for protected and/or covered corners (e.g., the fourth corner 176 and/or the third corner 174) and/or trailing corners (e.g., the second corner 172 and/or the third corner 174) when inserted into the first body 202 of the shell 200.

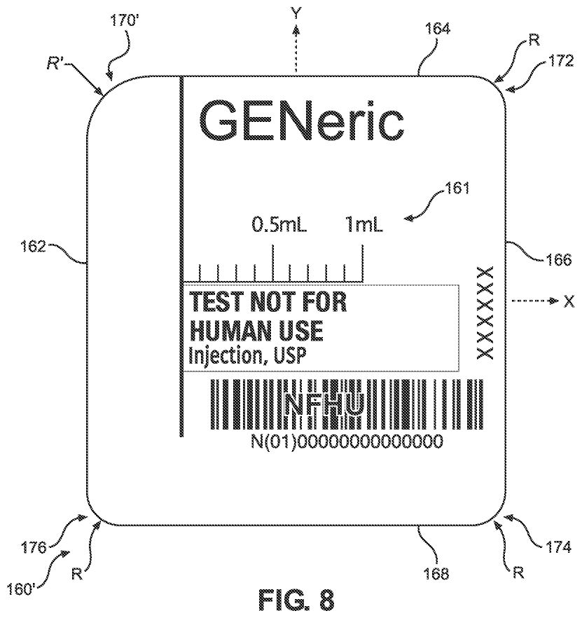

FIG. 8 illustrates a second embodiment of the film 160′. The disclosure of the first embodiment of the film 160 is incorporated herein with reference to the second embodiment of the film 160′ for sake of brevity, unless otherwise indicated. As illustrated, the first corner 170′ may have a rounded shaped but still different than the shape of the at least two of the other corners 172-176. For example, the shape of the first corner 170′ may be defined by a radius of curvature R′ greater than the radius of curvature R of the at least two of the other corners 172-176. As further illustrated, the radius of curvature R′ of the first corner 170′ may be greater than the radius of curvature R of each of the other corners 172-176. For example, the radius of curvature R′ of the first corner 170′ may be about 3 mm to about 9 mm, about 4 mm to about 8 mm, about 5 mm to about 7 mm, or about 6 mm. The radius of curvature R of at least two or more of the other corners 172-176 may be about 1 mm to about 5 mm, about 2 mm to about 4 mm, or about 3 mm. In other embodiments (not shown), the two or more or each of the corners 172-176 may form a sharp angle, for example 90 degree angles.

The film 160 may be produced by initially forming the film 160 with each of the corners 170-176 having the same shape and cutting the first corner 170, 170′ to produce its different shape (e.g., beveled or rounded).

The present disclosure is also directed to a method of assembling the system 10. The method may include inserting the tip 108 of the container body 102 into the adapter 140, and securing the plug 150 to close the container body 102 at the tip 108. The film 160 may be attached to the container body 102 and may secure the adapter 140 to the container body 102 and, in some embodiments the plug 150 to the adapter 140. The film 160 may be attached to the container body 102 by heat shrinking and/or an adhesive. In some embodiments, the film 160 may have a corner 170, 170′ that is beveled or rounded. The container body 102 may be inserted into the shell 200 with the corner 170, 170′ being a leading corner. The method further includes filling the container body 102 with the pharmaceutical material, and inserting the plunger rod 104 into the container body 102. In some embodiments, the method further includes placing the filled container 100 into a secondary package, such as a shell 200.

It will also be appreciated by those skilled in the art that modifications can be made to the example embodiments described herein without departing from the invention. Structural features of systems and apparatuses described herein can be replaced with functionally equivalent parts or omitted entirely. Moreover, it will be appreciated that features from the embodiments can be combined with each other without departing from the disclosure.

What is claimed is:

1. A injection device comprising:
a container body configured to contain a pharmaceutical material; and
a film disposed over the container body, wherein the film has a beveled corner.

2. The injection device of claim 1, wherein the film is a label having indicia pertaining to the pharmaceutical material.

3. The injection device of claim 1, wherein the film is heat shrunk over the container body.

4. The injection device of claim 1, wherein the container includes a plunger rod extending through the container body and being configured to push the pharmaceutical material out of the container body.

5. The injection device of claim 1, further comprising an adapter configured to connect to an external device, wherein the film extends over a portion of the adapter to fix the adapter to the container body.

6. The injection device of claim 5, wherein the container body includes a tip that extends through the adapter, and the adapter has a threaded inner surface.

7. The injection device of claim 5, wherein the container body is made of a glass and the adapter is made of a plastic.

8. The injection device of claim 5, further comprising a plug attached to the adapter and configured to seal a tip of the container body.

9. The injection device of claim 8, wherein the film extends over the plug.

10. The injection device of claim 1, wherein the film is asymmetric relative to at least one of a vertical axis and/or a lateral axis.

11. The injection device of claim 1, wherein the beveled corner extends at an angle of about 30 degrees to about 45 degrees relative to an axis of the film.

12. The injection device of claim 1, wherein the beveled corner has a length of about 6 mm to about 10 mm.

13. The injection device of claim 1, wherein the beveled corner extends at an angle of about 45 degrees relative to a vertical axis of the film and has a length of about 8 mm.

14. The injection device of claim 1, wherein the beveled corner limits or prevents peeling and/or curling of the film.

15. A system comprising:
the injection device of claim 1; and
a shell receiving the container.

16. The system of claim 15, wherein the shell is tubular.

17. The system of claim 15, wherein the shell includes a first body, a second body, and a frangible portion attaching the first body and the second body.

18. The system of claim 17, wherein the frangible portion is configured to be broken by rotating the first body relative to the second body.

19. The system of claim 15, wherein the container body is inserted into the shell with the beveled corner being a leading corner.

20. A system comprising:
a container including:
a container body containing a pharmaceutical material, and
a film disposed over the container body, wherein the film is asymmetric relative to at least one of a vertical and/or a lateral axis; and
a tubular shall receiving the container.

21. The system of claim 20, wherein the film has a first corner is rounded with a first radius of curvature and at least two second corners are rounded with a second radius of curvature, wherein the first radius of curvature is different than the second radius of curvature.

22. The system of claim 21, wherein the first radius of curvature is about 6 mm.

23. The system of claim 20, wherein the film has a beveled corner.

24. The system of claim 23, wherein the beveled corner extends at an angle of about 30 degrees to about 45 degrees relative to an axis of the film.

25. The system of claim 23, wherein the beveled corner has a length of about 6 mm to about 10 mm.

26. A system comprising:
a container including:
a container body containing a pharmaceutical material and having a tip,
a heat shrink film disposed over at least a portion of the container body, and
a plunger rod extending through the container body and being configured to push the pharmaceutical material out of the container body;
an adapter configured to connect to an external device, wherein the heat shrink film extends over the adapter to fix the adapter to the container body;
a plug received in the adapter and sealing the tip of the container body; and
a tubular shell receiving the adapter and the container,
wherein the heat shrink film has a beveled corner configured to limit or prevent peeling and/or curling, and the beveled corner extends at an angle of about 45 degrees relative to an axis of the heat shrink film and has a length of about 8 mm.

27. The system of claim 26, wherein the heat shrink film is asymmetric relative to at least one of a vertical and/or a lateral axis.

28. The system of claim 26, wherein the heat shrink film extends over the plug to fix the plug to the adapter.

29. A method of assembly comprising:
attaching a film to a container body, wherein the film has a beveled corner;
filling the container body with a pharmaceutical material;
inserting a plunger rod into the container body; and
inserting the container body into a shell with the beveled corner being a leading corner.

30. The method of claim 29, wherein attaching the film to the container body includes heat shrinking the film.

31. The method of claim 29, wherein attaching the film to the container body includes securing an adhesive to the container body.

32. The method of claim 29, further comprising fixing an adapter around a tip of the container body by attaching the film to the adapter.
